(19) United States
(12) Patent Application Publication
(10) Pub. No.: US 2007/0116770 A1
(43) Pub. Date: May 24, 2007

(54) Tazobactam/piperacillin lyophilizate

(76) Inventors: Christian Garms, Hamburg (DE); Klaus Glanzer, Hamburg (DE); Sonja Lieb, Hamburg (DE)

Correspondence Address: SMITH PATENT CONSULTING CONSULTING, LLC, 3309 DUKE STREET, ALEXANDRIA, VA, 22314, US

(21) Appl. No.: 11/392,825

(22) Filed: Mar. 30, 2006

(30) Foreign Application Priority Data
Nov. 22, 2005 (EP) EP 05.025448.1

(51) Int. Cl. A61K 31/43; A61K 9/14

(52) U.S. Cl. 424/489; 514/192

(57) ABSTRACT

The present invention relates to a lyophilizate which contains tazobactam and piperacillin, a process for the preparation of this lyophilizate, the use of this lyophilizate for the preparation of a drug, and drugs which comprise this lyophilizate.

The invention relates to a lyophilizate comprising tazobactam and piperacillin or the pharmaceutically tolerated salts thereof, a process for the preparation of this lyophilizate, the use thereof for the preparation of a drug, and drugs which comprise the lyophilizate.

Piperacillin is one of the acylaminopenicillins which are used, for example, in hospitals for the parenteral treatment of moderate and severe infections by gram-positive or gram-negative bacteria. As a result of excessive and incorrect administration of antibiotics infections by resistant bacterial strains have been occurring with increasing frequency in recent years and can no longer be treated by the use of normally used penicillins. The most frequent cause of bacterial resistance, in particular to acylaminopenicillins, is the formation of beta-actamases. These beta-lactamases are secreted by the resistant bacteria and destroy the penicillin active substances before they can control the bacteria. Inter alia, combination preparations of various penicillin antibiotics with beta-lactamase inhibitors are administered in order to solve this problem. By means of this, the resistance effect can be counteracted and the range of application of these penicillin antibiotics can be extended. Tazobactam is an Inhibitor for beta-lactamases which, in combination with certain penicillin derivatives, in particular, piperacillin, has an excellent effect. Tazobactam, like other beta-lactamase inhibitors, binds to the active center of the beta-lactamases. As a result, the catalytic center of these enzymes is destroyed and the Inhibitor is cleaved. Tazobactam has a high affinity to a broad spectrum of bacterial beta-lactamases. In particular, tazobactam binds to beta-lactamases from gram-positive and gram-negative as well as aerobic and anaerobic pathogens.

Tazobactam (a) and piperacillin (b) have the following structural formulas.

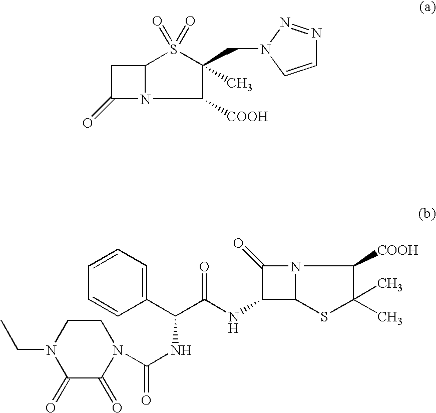

The active substances are available as a mixture under the trade names Zosyn® and Tazobac®, respectively, for Intravenous administration for the treatment of moderate to severe infections in injection vials in a customary dose of, for example, 2.5 g or 4.5 g or tazobactam/piperacillin mixtures, the mixing ratio being 2 g or 4 g of piperacillin to 0.5 g of tazobactam. Owing to the Instability of piperacillin in aqueous solution at room temperature, the active substances are present in solid form in the injection vials. Before their administration, the active substances must therefore be dissolved in a suitable solvent.

Tazobactam as a substance is disclosed in U.S. Pat. No. 4,562,073 among a number of penicillin derivatives. The publication also mentions the lyophilization of tazobactam as a monopreparation. A lyophilization process for piperacillin is disclosed in U.S. Pat. No. 4,477,452 and U.S. Pat. No. 4,534,977. These documents relate to problems with the dissolution of the active substance in an injection vial and propose a particularly porous lyophilizate of low density for solving this problem.

A combination preparation of piperacillin and tazobactam Is disclosed in U.S. Pat. No. 6,900,184. The object of this document is to minimize the particle formation during the reconstitution or the thawing of the combination preparation. This object is said to be achieved by addition of an aminocarboxylic acid as a chelating agent The process for the preparation of dry mixtures of active substances is regarded In this US patent as being not essential, but lyophilization of a solution which is said to contain 150-500 mg/ml of piperacillin and 15-125 mg/ml of tazobactam being proposed. Lower concentrations are discouraged owing to alleged disadvantages due to increasing production time and associated costs. In the example of this US patent, about 16.9 ml of an aqueous solution comprising about 235 mg/ml of piperacillin and about 30 mg/ml of tazobactam are Introduced Into ampoules and lyophilized.

A lyophilization process which leads to a lyophilizate of a galactomannan-free mixture of tazobactam and piperacillin is disclosed in WO 2005/074925.

A problem in the production of corresponding pharmaceutical preparations lies in the particular physicochemical properties of the lyophilizate, which is not flowable, is strongly hygroscopic and has an electrostatic charge, in particular of the fine Lyophilizate dust. Consequently, the lyophilizate cannot be introduced as a powder into vials, as is otherwise usual for cost reasons. Rather, as described in U.S. Pat. No. 6,900,184 the sterile-filtered solution of the active substances is itself introduced into vials and the solvent is then removed by freeze-drying. The filling of powder can be circumvented in this way, but with the disadvantage of additional high costs in the lyophilization process, which is in any case expensive. The utilization of the freeze-drying capacity with the use of vial lyophilization is poorer than that of bulk lyophilization by a factor of three, i.e. the production costs of vial freeze-drying are three times as high.

The direct mixing of the unlyophilized, separately precipitated active substances piperacillin sodium and tazobactam sodium is also not expedient. In addition to the very high tendency to strong cohesion due to the hygroscopic nature of the powders, and associated inhomogeneities, the rate of dissolution of the mixture thus prepared is in fact much slower than that of the mixture lyophilized in the vial, which usually dissolves In less than 10 seconds during the reconstitution of the injection. This gives rise to the risk of undissolved particles of substance in the ready-to-use solution for injection.

The mixing of previously lyophilized individual active substances might solve the problem of the dissolution rate. Technically, this possibility is not practicable owing to the physical properties of the very fine, amorphous powders and the associated problems, for example of inhomogeneities of the mixture owing to poor flowability of the powders, electrostatic charge buildups and hygroscopic nature of the powders.

An object of the present invention is therefore to provide a mixture of solid piperacillin and solid tazobactam, which mixture does not have the problems of the prior art. The mixture should be suitable for industrial further processing, In particular for dry filling.

Surprisingly, it was found that the colyophilized tazobactam/piperacillin is obtained as an amorphous, lumpy cake if the initial concentration of the solution at the beginning of the freeze-drying is <100 mg/ml, based on the total amount of the two free active substances. After sieving, for example over a continuously operating cone sieving machine (e.g. Glatt sieve G S, Frewitt T C or Quadro Comil, cf. W. A. Ritschel, A. Bauer-Brandl. “Die Tablette” [The Tablet], page 230 et seq.; 2nd edition, Editio Cantor Verlag, Aulendorf) having a sieve insert (hole size from 0.5 to 2.5 mm, preferably from 0.8 to 1.2 mm), this bulk lyophilizate gives a cohesion-free powder which consists of compact, dense amorphous particles. This amorphous, very free-flowing powder is outstandably suitable for the subsequent filling into vials. The lyophilizate thus obtained, which is suitable for filling as single doses, can be characterized by its specific extrusion volume.

The present invention therefore relates to a lyophilizate comprising tazobactam and piperacillin or the pharmaceutically tolerated salts thereof, which lyophilizate has a specific extrusion volume of >200 mm³/g.

The lyophilizate according to the invention is cohesion-free, free-flowing, suitable for filling as single doses and capable of bulk storage and transport. In addition, the lyophilizate can be handled in all production aspects without further additives.

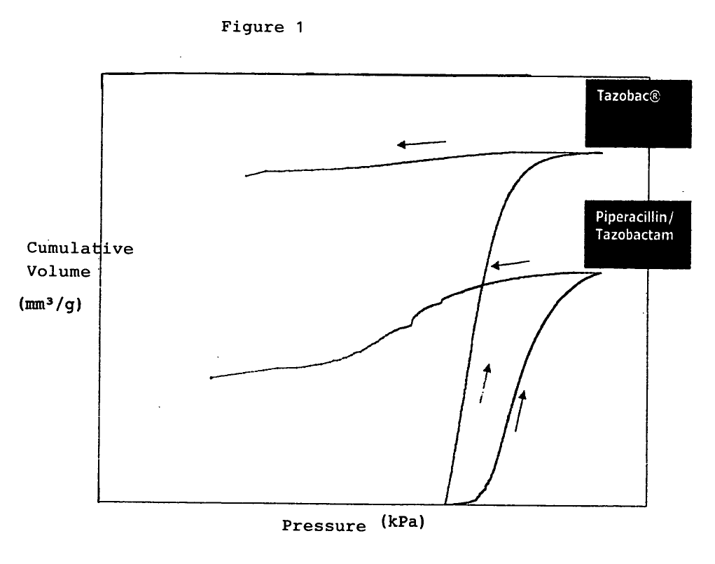

FIG. 1 shows the results of the low-pressure mercury porosimetry investigations for a comparative example (Tazobac®) and the lyophilizate according to the invention.

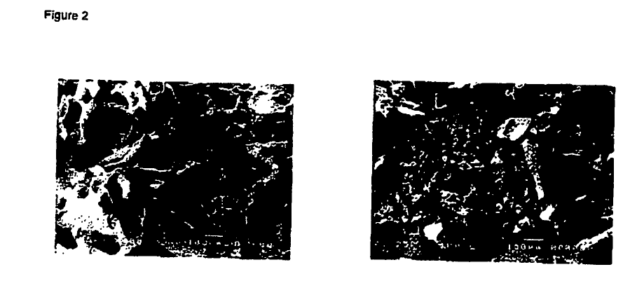

FIG. 2 shows scanning electron micrographs of lyophilizates under one hundred times magnification, of the commercially available comparative example Tazobac® on the left, which was lyophilized in the vial, and of the lyophilizate according to the invention on the right.

The specific extrusion volume of the lyophilizate according to the invention can be determined by low-pressure mercury porosimetry. According to the invention, the specific extrusion volume was determined using a Pascal 140 (Thermoquest I). For the pressure build-up and pressure decline procedure, an average speed was chosen (dP=5; dN 5). The pore volume of the sample was obtained by capacitive measurement (resolution: 0.1 mm³). Pressure and volume were recorded with high resolution both during pressure build-up and during pressure decline. The specific extrusion volume is calculated from the difference between final volume and total cumulative volume and the weight of sample taken. It is a measure of the powder aggregation. Further details of the method described are to be found in “Hg-Niederdruckporosimetrie [Hg low-pressure porosimetry]: Pascal Series Brochure. Thermo Electron Corporation”.

By means of the method described above, the specific extrusion volume of both a lyophilizate commercially available under the trade name Tazobac® and the lyophilizate according to the invention was determined. The results of the measurement are shown In FIG. 1. The two lyophilizates differ in the initial concentration of the solution at the beginning of the freeze-drying. It is evident that mercury intrudes substantially more rapidly and also in a higher volume into the highly porous sample of Tazobac® than in the case of the lyophilizate according to the invention. In the case of Tazobac® the cumulative volume was 1646 mm³/g whereas the cumulative volume was only 1089 mm³/g in the case of the lyophilizate according to the invention. The specific extrusion volume for Tazobac® was determined as 121 mm³/g, and the specific extrusion volume of the lyophilizate according to the invention was determined as 508 mm³/g.

According to the Invention, the lyophilizate thus has a specific extrusion volume of >200 mm³/g, preferably of >300 mm³/g, in particular of >400 mm³/g and particularly preferably of about 500 mm³/g.

For the further characterization of the lyophilizate according to the invention, scanning electromicrographs were prepared. The results are shown in FIG. 2. The Tazobac® sample not according to the invention (left in FIG. 2) has an appearance typical for lyophilizates. Surprisingly, the lyophilizate according to the invention has a completely different appearance, the lyophilizate containing fewer porous particles having polygonal surfaces. The lyophilizate according to the invention therefore has substantially better flowabilty than the conventional lyophilizate.

Preferably, the lyophilizate according to the invention also has a true density of >1.38 g/cm³, in particular of >1.40 g/cm³ and particularly preferably of about 1.42 g/cm³. The true density can be determined as explained in more detail In the example below. Further details are to be found in the technical brochure “Pycnometers” from Quantochrome.

In a further preferred embodiment the lyophilizate according to the invention has a D90 cumulative undersize parameter of <70 μm, preferably in the range of 55-65 μm. Moreover, the D10 cumulative undersize parameter may be <8 μm, preferably in the range of 2-6 μm and the D50 cumulative undersize parameter may be <35 μm, in particular in the range of 25-35 μm. The measurement of the cumulative undersize parameters is explained in more detail in the example below. Further details are to be found in the technical brochure “Laser Particle Sizer” from Fritsch.

The lyophilizate according to the invention should dissolve in less than 10 seconds, in a customary dose which can be administered for injection, in a solvent customary for injections. It therefore has a dissolution rate comparable to a mixture lyophilized in the vial.

Suitable pharmaceutically tolerated salts of the active substances are all salts known to the person skilled in the art. Preferably, both the tazobactam and the piperacillin are used in the form of the sodium salt. In a preferred embodiment, the lyophilizate according to the invention contains no aminocarboxylic acid chelator or a pharmaceutically tolerated salt thereof. In a particularly preferred embodiment the lyophilizate contains, apart from a buffer if appropriate, no further additives, such as, for example, diluents or agents for promoting the dissolution rate.

The weight ratio of tazobactam to piperacillin in the lyophilizate according to the invention can be chosen by the person skilled in the art in accordance with the desired use. The ratio may be, for example, in the range from 1:2 to 1:10, preferably about 1:4 or about 1:8.

The lyophilizate according to the invention is obtained as an amorphous lumpy cake. After sieving, this bulk lyophilizate gives a cohesion-free powder which consists of compact, densely amorphous particles. In contrast to conventional lyophilizate, this powder is suitable for subsequent filling into vials. In addition, it is capable of bulk storage and transport.

In addition, it has surprisingly been found that the lyophilizate according to the invention is less hygroscopic than conventional lyophilizates. This has an additional positive effect on the filling properties of the product.

The lyophilizate according to the invention consists of a homogeneous mixture of the active substances. In contrast, separately lyophilized powders comprising active substance are difficult to combine to give a homogeneous mixture since they have different particle densities and the build up of electrostatic charges results. The lyophilizate according to the invention therefore solves the problems of the lyophilizates of the prior art.

The lyophilizate according to the invention can be prepared by freeze-drying of an aqueous solution of tazobactam and piperacillin or the pharmaceutically tolerated salts thereof, the initial concentration of the solution at the beginning of the freeze-drying being <100 mg/ml, based on the total amount of the two free active substances. Preferably, the initial concentration is <70 mg/ml, particularly preferably <50 mg/ml. The individual concentrations of the two active substances can be chosen so that the ratio corresponds to the desired ratio in the subsequent dosage form. Accordingly, the initial concentration of piperacillin may be, for example, about 40 mg/ml or about 20 mg/ml and the initial concentration of tazobactam may be about 5 mg/ml. The preferred initial concentration of the solution at the beginning of the freeze-drying is accordingly in the range of 20-50 mg/ml, in particular 25-45 mg/ml, based on the total amount of the two free active substances. Owing to the comparatively low concentration of the active substances, the process according to the invention is preferably not carried out in small vials or ampoules which simultaneously serve for storing a unit dose of the active substances, but in larger containers which permit a larger initial volume of the solution to be lyophilized, such as, for example, an initial volume of a few liters, e.g. >10 liters, preferably about 50 liters.

Preferably, in the process according to the invention, a solution which has a pH in the range from 5.0 to 7.0, in particular about 6.0, is freeze-dried. The pH of the solution can be adjusted, for example, with sodium bicarbonate. The solution may be an aqueous solution. The other process parameters for the lyophilization can be freely chosen by the person skilled in the art according to his technical knowledge. Suitable process parameters are to be found, for example, in WO 2005/074925, the content of which is therefore incorporated by reference in the present Application.

The present invention also relates to a lyophilizate which is obtainable by the process described above, the use of the lyophilizate according to the invention for the preparation of a drug, and drugs which comprise a corresponding lyophilizate. Preferably, the lyophilizate according to the invention, optionally after further processing, such as, for example, sieving, is introduced for this purpose in the desired amount of, for example, about 2.5 g or about 4.5 g based on the free active substances, into ampoules. Shortly before injection, the solution is then reconstituted by adding a suitable solvent.

The present invention will now be explained in more detail by the following example which is not to be understood as being limiting.

### EXAMPLE

50 l of water qs were introduced into a stainless steel reactor and cooled, 250 g of tazobactam and 2 kg of piperacillin were weighed in and thoroughly mixed. The pH of the mixture was adjusted to 6.0 with sodium bicarbonate. In a sterile environment, the solution was first filtered, then sterile-filtered and lyophilized. The lyophilization cake was then milled.

The specific extrusion volume of the lyophilizate obtained as described above was determined by means of a Pascal 140 (Thermoquest, I) by means of a low-pressure mercury measurement (0.01-350 kPa). For the pressure build-up and pressure decline procedure an average speed was chosen (dP=5; dN=5). The pore volume of the sample was measured by a capacitive method (resolution; 0.1 mm³). Pressure and volume were recorded with high resolution both during the pressure build-up and during the pressure decline. The commercially available Tazobac® product lyophilized in vials was used as a comparative substance. The results of the measurement are shown In the attached FIG. 1 and are summarized in table 1 below.

TABLE 1

| | Specific extrusion volume | Cumulative volume |
|---|---|---|
| Sample | [mm³/g] | [mm³/g] |
| Tazobac ® | 121 | 1646 |
| Lyophilizate according | 508 | 1089 |
| to the invention | | |

For the SEM investigations, the samples were applied to a carbon paddy and sputtered with gold. Scanning electron micrographs were recorded In the low vacuum mode (about 8 Pa). Recordings at 100 times magnification both for Tazobac® and for the lyophilizate according to the invention are shown in FIG. 2.

The true density of the samples was determined by means of a Quantachrome Ultrapyknometer 1000. The spray and measuring gas was helium. In the case of two parallel analyses In each case (n=6) a mean value of 1.360 g/cm³ was found for the true density of Tazobac® and a mean value of 1.421 g/cm³ for the lyophilizate according to the invention.

The particle distribution of the samples was determined by means of a Fritsch Analysette 22 Compact (measuring range 0.3-300 μm). The circulation pump and also the dispersing unit was a Fritsch “Apollo”. The deagglomeration was effected with the aid of an ultrasonic rod (Dr. Hilscher GmbH, ultrasonic processor UP200H) with parameters: 60: 0.5; time: 15 sec.

This constitutes wet dispersing In an inert silicone oil dispersant (Wacker AK10). The sample was deagglomerated in a 50 ml beaker with the addition of about 25 ml of dispersant with the aid of the ultrasonic rod and immediately thereafter fed to the circulation pump. The particle measurement started automatically from a beam absorption of 12%. The background measurement was activated and 7 scans/measurements were carried out. The circulation pump was cleaned after each measurement and filled with fresh silicone oil. 3 analyses were carried out in each case, the averaged values of the cumulative undersize parameters of which are summarized In table 2 below:

TABLE 2

| Cumulative undersize | | Lyophilizate according to the |
|---|---|---|
| parameter | Tazobac ® | invention |
| D10 [μm] | 10.946 | 4.820 |
| D50 [μm] | 41.559 | 29.874 |
| D90 [μm] | 82.712 | 61.781 |

Finally, the water content of the samples was determined by means of Karl Fischer titration. A Mettler Toledo DL38 titrator was used for the analysis. At the beginning, a concentration determination was carried out with oxalic acid and then the samples were analyzed (n=3). The results are summarized in table 3 below.

TABLE 3

| | |
|---|---|
| | Lyophilizate |
| | according to the |
| Tazobac ® | invention |

| Conditioning | As is | 43% RH | As is | 43% RH |
|---|---|---|---|---|
| 1st analysis | 1.88% | 4.69% | 1.68% | 4.11% |
| 2nd analysis | 1.73% | 4.65% | 1.85% | 4.12% |
| 3rd analysis | 1.70% | 4.53% | 1.56% | 4.15% |
| Mean value | 1.77% | 4.63% | 1.70% | 4.13% |

It is evident that both samples initially have a similar water content. Without preconditioning (i.e. the samples were taken directly from a previously closed container), the values are about 1.7% of water (based on moist starting substance). After conditioning at 43% relative humidity (RH) over 15 hours, the water content in Tazobac® increased to 4.63% whereas the water content in the lyophilizate according to the invention increased to only 4.13%. The lyophilizate according to the invention is therefore less hygroscopic than the conventional lyophilizate.

1. A lyophilizate comprising tazobactam and piperacillin or the pharmaceutically tolerated salts thereof, wherein the lyophilizate has a specific extrusion volume of >200 mm³/g.

2. The lyophilizate of claim 1, which has a true density of >1.38 g/cm³.

3. The lyophilizate of claim 1, which has a D90 cumulative undersize parameter of <70 μm.

4. The lyophilizate of claim 1, which is particulate and flowable.

5. The lyophilizate of claim 1, which dissolves in less than 10 seconds in a customary dose which can be administered for injection.

6. The lyophilizate of claim 1, wherein the tazobactam and piperacillin are present in the form of the sodium salts thereof.

7. The lyophilizate of claim 1, which comprises no aminocarboxylic acid chelator or pharmaceutically tolerated salt thereof.

8. The lyophilizate of claim 1, wherein the weight ratio of tazobactam to piperacillin is in the range from 1:2 to 1:10.

9. A process for the preparation of the lyophilizate of claim 1, comprising the step of freeze-drying of an aqueous solution of tazobactam and piperacillin or the pharmaceutically tolerated salts thereof, wherein the initial concentration of the solution at the beginning of the freeze-drying is <100 mg/ml, based on the total amount of the two free active substances.

10. The process of claim 9, wherein the pH of the solution is in the range from 5.0 to 7.0.

11. A lyophilizate obtained by the process of claim 9.

12. A method of preparing a drug comprising the step of dissolving lyophilizate of claim 1 in solvent customary for injections.

13. A drug comprising the lyophilizate of claim 1.

14. The lyophilizate of claim 1, wherein the weight ratio of tazobactam to piperacillin is in the range from 1:4 to 1:8.

15. The lyophilizate of claim 1, wherein the weight ratio of tazobactam to piperacillin is about 1:8.

16. The process of claim 9, wherein the pH of the solution is about 6.0.
